(19) United States
(12) Patent Application Publication
(10) Pub. No.: US 2026/0356786 A1
(43) Pub. Date: Nov. 12, 2026

(54) METHOD AND COMPOSITION TO REMOVE POLYMER FOULANT

(71) Applicant: ECOLAB USA Inc., St. Paul, MN (US)

(72) Inventor: Meng KONG, Shanghai (CN)

(73) Assignee: ECOLAB USA Inc., St. Paul, MN (US)

(21) Appl. No.: 18/713,139

(22) PCT Filed: Dec. 2, 2022
(86) PCT No.: PCT/US2022/051667

§ 371 Date: May 23, 2024

(30) Foreign Application Priority Data
Dec. 8, 2021 (CN) 202111489084.6

(51) Int. Cl. C07C 51/50; C11D 7/50

(52) U.S. Cl. CPC C07C 51/50; C11D 7/5013; C11D 7/5022; C11D 7/5027

(57) ABSTRACT

A composition and method for dissolving foulant formed in a monomer manufacturing process. The composition may include an amide-containing compound and a solvent. Other components may optionally be included in the composition, such as a polymerization inhibitor or dispersant. The amide-containing compound may be, for example, dimethylacetamide. The solvent may be, for example, methanol or toluene. The foulant includes a polymer.

### TECHNICAL FIELD

The present disclosure generally relates to monomer manufacturing processes. More particularly, the disclosure relates to methods and compositions for dissolving polymeric foulants produced during monomer manufacturing processes.

### BACKGROUND

Polymerization of methyl methacrylate, methacrylic acid, methacrylamide or other monomers is undesirable but very common during processes for producing these monomers. In the methyl methacrylate manufacturing process, polymers formed from methyl methacrylate, methacrylic acid, and other monomers flow out of the process with the spent medium. Many of the polymers formed have a lower density than the spent medium, so they float and can deposit on the equipment, which can result in operational problems.

A common method used to combat polymer fouling involves the use of inhibitors to inhibit the formation of oligomers. Methods may also include applying a dispersant to the medium. However, these methods have limited effect when the amount of polymer in the medium is high. While these methods of preventing fouling are effective to varying degrees, they can be time consuming and/or costly to implement. Further, their efficiencies at removing or preventing fouling tend to be low.

### BRIEF SUMMARY

The disclosure provides methods and compositions for dissolving polymer foulants formed during monomer manufacturing processes.

In some embodiments, the present disclosure provides a method of dissolving a polymer in a monomer manufacturing process. The method comprises adding a composition to a medium in the process, wherein the composition comprises an amide-containing compound and wherein the medium comprises the polymer. The method further comprises contacting the amide-containing compound with the polymer, and dissolving the polymer.

In some embodiments, the amide-containing compound comprises the following formula:

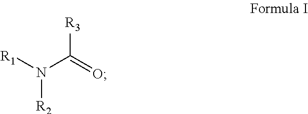

wherein R₁, R₂, and R₃ are independently selected from hydrogen, an alkyl group, an alkenyl group, and an aryl group. In some embodiments, at least two of R₁, R₂, and R₃ are CH₃.

In certain embodiments, the amide-containing compound comprises the following formula:

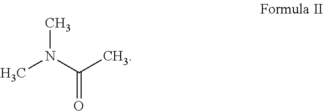

In some embodiments, the amide-containing compound has a weight average molecular weight from about 45 g/mol to about 300 g/mol.

In certain embodiments, the composition comprises at least two different amide-containing compounds.

In some embodiments, the monomer is selected from the group consisting of acrylic acid, methacrylic acid, an acrylate, a methacrylic ester, acrylonitrile, and any combination thereof.

In some embodiments, the composition further comprises a solvent. The solvent may be selected from, for example, a hydrocarbon, an ether, an ester, an alcohol, tetrahydrofuran, and any combination thereof.

In certain embodiments, the solvent is selected from the group consisting of a hydrocarbon, tetrahydrofuran, an ether, and any combination thereof, and the monomer is acrylic acid.

In certain embodiments, the solvent is selected from the group consisting of an alcohol, a hydrocarbon, and any combination thereof, and the monomer is selected from the group consisting of a methacrylic ester, an acrylate, and any combination thereof.

In some embodiments, the hydrocarbon comprises from about 5 to about 20 carbon atoms. In certain embodiments, the hydrocarbon is selected from toluene, kerosene, or any combination thereof.

In some embodiments, the composition comprises about 10 wt. % to about 90 wt. % of the amide-containing compound and about 90 wt. % to about 10 wt. % of the solvent.

In some embodiments, a component of the monomer manufacturing process comprises the medium. In certain embodiments, the component is selected from the group consisting of an absorption column, a recovery column, a purification column, a stripper, a product column, a reboiler, a spent acid tank, a conduit connecting any of the foregoing components, and any combination thereof.

In some embodiments, the composition is added continuously or intermittently to the medium.

In some embodiments, the composition is combined with an alkali wash water in a weight ratio of about 1:10 to about 1:1 and added to the medium.

In certain embodiments, the composition and dissolved polymer are removed from the medium.

In some embodiments, about 10 ppm to about 10,000 ppm of the amide-containing compound is added to the medium. In some embodiments, the medium comprises from about 0.5 wt. % to about 5 wt. % of the amide-containing compound.

In certain embodiments, the composition further comprises a polymerization inhibitor and/or a dispersant.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### BRIEF DESCRIPTION OF THE DRAWINGS

A detailed description of the invention is hereafter described with specific reference being made to the drawings in which:

FIG. 1 shows a schematic of an embodiment of the present disclosure related to a water separation column; and

FIG. 2 shows a schematic of an embodiment of the present disclosure related to a spent acid tank.

### DETAILED DESCRIPTION

Various embodiments are described below, some with reference to the drawings. The relationship and functioning of the various elements of the embodiments may better be understood by reference to the following detailed description. However, embodiments are not limited to those illustrated in the drawings and/or explicitly described below. It should be understood that the drawings are not necessarily to scale, and in certain instances, details may have been omitted that are not necessary for an understanding of embodiments disclosed herein.

Unless otherwise indicated, an alkyl group as described herein alone or as part of another group is an optionally substituted linear or branched saturated monovalent hydrocarbon substituent containing from, for example, one to about sixty carbon atoms, such as one to about thirty carbon atoms, in the main chain. Examples of unsubstituted alkyl groups include methyl, ethyl, n-propyl, i-propyl, n-butyl, i-butyl, s-butyl, t-butyl, n-pentyl, i-pentyl, s-pentyl, t-pentyl, and the like.

The terms “aryl” or “ar” as used herein alone or as part of another group (e.g., arylene) denote optionally substituted homocyclic aromatic groups, such as monocyclic or bicyclic groups containing from about 6 to about 12 carbons in the ring portion, such as phenyl, biphenyl, naphthyl, substituted phenyl, substituted biphenyl or substituted naphthyl. The term “aryl” also includes heteroaryl functional groups. It is understood that the term “aryl” applies to cyclic substituents that are planar and comprise 4n+2n electrons, according to Huckel's Rule.

“Cycloalkyl” refers to a cyclic alkyl substituent containing from, for example, about 3 to about 8 carbon atoms, preferably from about 4 to about 7 carbon atoms, and more preferably from about 4 to about 6 carbon atoms. Examples of such substituents include cyclopropyl, cyclobutyl, cyclopentyl, cyclohexyl, cycloheptyl, cyclooctyl, and the like. The cyclic alkyl groups may be unsubstituted or further substituted with alkyl groups, such as methyl groups, ethyl groups, and the like.

Compounds of the present disclosure may be substituted with suitable substituents. The term “suitable substituent,” as used herein, is intended to mean a chemically acceptable functional group, preferably a moiety that does not negate the activity of the compounds. Such suitable substituents include, but are not limited to, halo groups, perfluoroalkyl groups, perfluoro-alkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkoxycarbonyl groups, alkylaminocarbonyl groups, dialkylamino carbonyl groups, arylcarbonyl groups, aryloxy-carbonyl groups, alkylsulfonyl groups, and arylsulfonyl groups. In some embodiments, suitable substituents may include halogen, an unsubstituted C₁-C₁₂ alkyl group, an unsubstituted C₄-C₆ aryl group, or an unsubstituted C₁-C₁₀ alkoxy group. Those skilled in the art will appreciate that many substituents can be substituted by additional substituents.

The term “substituted” as in “substituted alkyl,” means that in the group in question (i.e., the alkyl group), at least one hydrogen atom bound to a carbon atom is replaced with one or more substituent groups, such as hydroxy (—OH), alkylthio, phosphino, amido (—CON(R_{A})(R_{B}), wherein R_{A} and R_{B} are independently hydrogen, alkyl, or aryl), amino (—N(R_{A})(R_{B}), wherein R_{A} and R_{B} are independently hydrogen, alkyl, or aryl), halo (fluoro, chloro, bromo, or iodo), silyl, nitro (—NO₂), an ether (—OR_{A} wherein R_{A} is alkyl or aryl), an ester (—OC(O)R_{A} wherein R_{A} is alkyl or aryl), keto (—C(O)R_{A} wherein R_{A} is alkyl or aryl), heterocyclo, and the like.

When the term “substituted” introduces a list of possible substituted groups, it is intended that the term apply to every member of that group. That is, the phrase “optionally substituted alkyl or aryl” is to be interpreted as “optionally substituted alkyl or optionally substituted aryl.”

The terms “polymer,” “copolymer,” “polymerize,” “copolymerize,” and the like include not only polymers comprising two monomer residues and polymerization of two different monomers together, but also include (co) polymers comprising more than two monomer residues and polymerizing together more than two or more other monomers. For example, a polymer as disclosed herein includes a terpolymer, a tetrapolymer, polymers comprising more than four different monomers, as well as polymers comprising, consisting of, or consisting essentially of two different monomer residues. Additionally, a “polymer” as disclosed herein may also include a homopolymer, which is a polymer comprising a single type of monomer unit.

Unless specified differently, the polymers of the present disclosure may be linear, branched, cross-linked, structured, synthetic, semi-synthetic, natural, and/or functionally modified. A polymer of the present disclosure can be in the form of a solution, a dry powder, a liquid, or a dispersion, for example.

The terms “foul” and “fouling” refer to a process of forming, adding, and/or depositing a layer of extraneous material on the surface of equipment.

The term “foulant” refers to a layer or deposit of extraneous material on the surface of equipment. A foulant may also be suspended, dissolved, and/or dispersed in the medium. Generally, the foulant may be formed from an agglomeration of polymers and may optionally include inorganic material.

The terms “dissolve” or “dissolving” mean the foulant is reduced from an agglomerated mass to polymer particles (groups of one or more polymers). The polymer particles may transition from solid phase to a liquid phase. In some aspects, dissolving includes degrading polymers and/or reducing their molecular weight. Once dissolved, the foulant can be removed, for example, through the process stream. In some embodiments, dissolving the agglomerated mass includes diffusing the polymer particles in the process stream.

The terms “wt. %”, “vol. %” or “mol. %” refer to a weight, volume, or molar percentage of a component, respectively, based on the total weight, the total volume, or the total moles of material that includes the component.

In an illustrative embodiment, a method of dissolving a polymer in a monomer manufacturing process is provided. The method comprises adding a composition to a medium in the process, wherein the composition comprises an amide-containing compound and wherein the medium comprises the polymer. The amide-containing compound contacts and dissolves the polymer.

Without being limited by theory, the process of dissolving the polymer is thought to occur as follows: the amide-containing compound and foulant formed from polymers of methyl methacrylate, acrylic acid, acrylonitrile, and the like have similar solubility properties. The foulant may resemble polymer chains tangled like balls of twine. First, the amide-containing compound will permeate into the foulant and loosen the molecular chains. Second, the loosened polymer and/or polymer particles will transition from solid phase to liquid phase. At the same time, the amide-containing compound will be released into the medium. Finally, the dissolved polymers and amide-containing compound may be removed, for example, by a process stream formed from the medium.

The presently disclosed methods and compositions are useful in connection with any process used to manufacture one or more reactive monomers. For example, if a manufacturing process produces one or more monomers that are capable of polymerizing or self-polymerizing, then the presently disclosed methods and compositions may be useful in such a process. In some aspects, the reactive monomer is selected from the group consisting of acrylic acid, methacrylic acid, an acrylate, a methacrylic ester, acrylonitrile, and any combination thereof.

### Composition

The compositions disclosed herein comprise an amide-containing compound. In some embodiments, the compositions may include a solvent. The amide-containing compound may be present in the composition at about 10 wt. % to about 100 wt. % of the composition and the solvent may be present at about 90 wt. % to about 0 wt. % of the composition. For example, the amide-containing compound may be present at about 10 wt. % to about 100 wt. %, about 15 wt. % to about 100 wt. %, about 20 wt. % to about 100 wt. %, about 25 wt. % to about 100 wt. %, about 30 wt. % to about 100 wt. %, about 35 wt. % to about 100 wt. %, about 40 wt. % to about 100 wt. %, about 45 wt. % to about 100 wt. %, about 50 wt. % to about 100 wt. %, about 55 wt. % to about 100 wt. %, about 60 wt. % to about 100 wt. %, about 65 wt. % to about 100 wt. %, about 70 wt. % to about 100 wt. %, about 75 wt. % to about 100 wt. %, about 80 wt. % to about 100 wt. %, about 85 wt. % to about 100 wt. %, about 90 wt. % to about 100 wt. %, or about 95 wt. % to about 100 wt. % of the composition. In some embodiments, the amide-containing compound is about 25 wt. % to about 75 wt. %, about 30 wt. % to about 70 wt. %, about 35 wt. % to about 65 wt. %, about 40 wt. % to about 60 wt. %, or about 45 wt. % to about 55 wt. % of the composition. In some embodiments, the amide-containing compound is about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 80 wt. %, about 85 wt. %, about 90 wt. %, about 95 wt. %, or about 100 wt. % of the composition.

In some embodiments, the composition comprises more than one amide-containing compound. For example, the composition may comprise at least two different amide-containing compounds. In some embodiments, the composition comprises three, four, five or more amide-containing compounds.

The composition may include a solvent. The solvent may be present at about 90 wt. % to about 0 wt. %, about 85 wt. % to about 0 wt. %, about 80 wt. % to about 0 wt. %, about 75 wt. % to about 0 wt. %, about 70 wt. % to about 0 wt. %, about 65 wt. % to about 0 wt. %, about 60 wt. % to about 0 wt. %, about 55 wt. % to about 0 wt. %, about 50 wt. % to about 0 wt. %, about 45 wt. % to about 0 wt. %, about 40 wt. % to about 0 wt. %, about 35 wt. % to about 0 wt. %, about 30 wt. % to about 0 wt. %, about 25 wt. % to about 0 wt. %, about 20 wt. % to about 0 wt. %, about 15 wt. % to about 0 wt. %, about 10 wt. % to about 0 wt. %, or about 5 wt. % to about 0 wt. % of the composition. In some embodiments, the solvent is present at about 95 wt. % to about 5 wt. %, about 90 wt. % to about 10 wt. %, about 85 wt. % to about 15 wt. %, about 80 wt. % to about 20 wt. %, about 75 wt. % to about 25 wt. %, about 70 wt. % to about 30 wt. %, about 65 wt. % to about 35 wt. %, about 60 wt. % to about 40 wt. %, or about 55 wt. % to about 45 wt. % of the composition. In some embodiments, the solvent may be present at about 0.001 wt. %, about 0.01 wt. %, about 0.1 wt. %, about 1 wt. %, about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, or about 90 wt. % of the composition.

In some aspects, the amide-containing compound has a weight average molecular weight from about 45 g/mol to about 300 g/mol, about 50 g/mol to about 275 g/mol, about 55 g/mol to about 250 g/mol, about 60 g/mol to about 225 g/mol, about 65 g/mol to about 200 g/mol, about 70 g/mol to about 175 g/mol, about 75 g/mol to about 150 g/mol, about 100 g/mol to about 125 g/mol, about 73 g/mol to about 270 g/mol, or about 73 g/mol to about 120 g/mol. In some embodiments, the amide-containing compound has a molecular weight of about 45 g/mol, about 50 g/mol, about 55 g/mol, about 60 g/mol, about 65 g/mol, about 70 g/mol, about 75 g/mol, about 80 g/mol, about 85 g/mol, about 90 g/mol, about 95 g/mol, about 100 g/mol, about 105 g/mol, about 110 g/mol, about 115 g/mol, about 120 g/mol, about 125 g/mol, about 130 g/mol, about 135 g/mol, about 140 g/mol, about 145 g/mol, about 150 g/mol, about 155 g/mol, about 160 g/mol, about 165 g/mol, about 170 g/mol, about 175 g/mol, about 180 g/mol, about 185 g/mol, about 190 g/mol, about 195 g/mol, about 200 g/mol, about 205 g/mol, about 210 g/mol, about 215 g/mol, about 220 g/mol, about 225 g/mol, about 230 g/mol, about 235 g/mol, about 240 g/mol, about 245 g/mol, about 250 g/mol, about 260 g/mol, about 265 g/mol, about 270 g/mol, about 275 g/mol, about 280 g/mol, about 285 g/mol, about 290 g/mol, about 295 g/mol, or about 300 g/mol. In some embodiments, the molecular weight of the amide-containing compound is about 45 g/mol, about 73 g/mol, about 120 g/mol, about 270 g/mol, or about 300 g/mol.

In some embodiments, the amide-containing compound comprises a structure of Formula I.

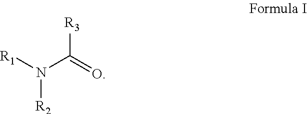

In some embodiments, R₁, R₂, and R₃ are independently selected from the group consisting of hydrogen, alkyl, alkenyl, and aryl. In some embodiments, at least one of R₁, R₂, or R₃ are CH₃. In another aspect, at least two of R₁, R₂, or R₃ are CH₃. In an illustrative embodiment, the amide-containing compound comprises a structure of Formula II.

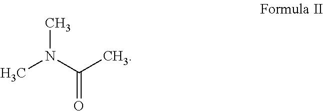

In another embodiment, the amide-containing compound is selected from the group consisting of dimethylformamide “DMF” (CAS No 68-12-2), dimethylacetamide “DMAC” (CAS 127-19-5), and N,N-diethylpropionamide (CAS 1114-51-8).

When R₁, R₂, or R₃ is an alkyl, the alkyl may include from one to sixty carbon atoms, one to fifty carbon atoms, one to forty carbon atoms, one to thirty carbon atoms, one to twenty-five carbon atoms, one to twenty carbon atoms, one to fifteen carbon atoms, one to ten carbon atoms, or one to five carbon atoms. In some embodiments, R₁, R₂, and/or R₃ is C₁ alkyl, C₂ alkyl, C₃ alkyl, C₄ alkyl, C₅ alkyl, C₆ alkyl, C₇ alkyl, C₈ alkyl, C₉ alkyl or C₁₀ alkyl.

When R₁, R₂, or R₃ is an alkenyl, the alkenyl may include from one to sixty carbon atoms, one to fifty carbon atoms, one to forty carbon atoms, one to thirty carbon atoms, one to twenty-five carbon atoms, one to twenty carbon atoms, one to fifteen carbon atoms, one to ten carbon atoms, or one to five carbon atoms. In some embodiments, R₁, R₂, and/or R₃ is C₁ alkenyl, C₂ alkenyl, C₃ alkenyl, C₄ alkenyl, C₅ alkenyl, C₆ alkenyl, C₇ alkenyl, C₈ alkenyl, C₉ alkenyl or C₁₀ alkenyl.

The solvent may be selected from the group consisting of a hydrocarbon, an ether, an ester, an alcohol, tetrahydrofuran, and any combination thereof. When the solvent includes a hydrocarbon, the hydrocarbon may comprise from about 5 to about 25 carbon atoms, about 5 to about 20 carbon atoms, about 5 to about 15 carbon atoms, or about 5 to about 10 carbon atoms. In some embodiments, the solvent comprises a hydrocarbon selected from toluene, kerosene, xylene, and any combination thereof.

In an illustrative embodiment, the solvent is selected from the group consisting of a hydrocarbon, tetrahydrofuran, an ether, and any combination thereof, and the monomer is acrylic acid. In another illustrative embodiment, the solvent is selected from the group consisting of an alcohol, a hydrocarbon, and any combination thereof, and the monomer is selected from the group consisting of a methacrylic ester, an acrylate, and any combination thereof.

### Method of Using the Composition

The compositions disclosed herein may be used in monomer manufacturing processes, for example, or any process that may include reactive monomers. The compositions may be added to a medium in the process. The medium comprises the foulant. The amide-containing compound of the composition contacts and dissolves the foulant.

The monomer manufacturing process may include a plurality of components and the component may include the medium. The composition may be added continuously to the medium. In some embodiments, the composition may be added intermittently to the medium. In certain embodiments, the composition may be added during a wash cycle of the manufacturing components. When added to a wash cycle, the composition may be combined with an alkali wash water to form a mixture having a weight ratio of about 1:10 (composition to wash) to about 1:1 before introduction into the manufacturing components. In some aspects, the composition and alkali wash water mixture is added to the medium.

The composition may be added to the medium manually, automatically, or a combination thereof. In some embodiments, the composition is added to the medium and/or directly to a component of the monomer manufacturing process. The composition may be injected into the medium and/or one or more components. The component may include the medium. As an example, the composition may be injected into a feed pipeline and/or a toluene pipeline. The medium may contain for example raw acrylic acid or a solvent.

In some embodiments, the component is selected from the group consisting of an absorption column, a stripper, a recovery column, a purification column, a product column, a reboiler, a spent acid tank, a feed pipeline, a toluene pipeline, a tray or trays in the foreruns, a conduit connecting any of the foregoing components, and any combination thereof.

The location for addition of the composition may be selected based on, for example, presence of the foulant or suspected presence of the foulant. In some embodiments, the composition may be added to a location in the monomer manufacturing process that is upstream of a location comprising a foulant. In some embodiments, the composition is added, for example, by direct injection into a location blocked by foulant.

The foulant may have a volume unsuitable for removal by a dispersant. In some embodiments, the foulant ranges in volume from about 0.5 mL³ to about 50 mL³, about 0.5 mL³ to about 45 mL³, about 0.5 mL³ to about 40 mL³, about 0.5 mL³ to about 35 mL³, about 0.5 mL³ to about 30 mL³, about 0.5 mL³ to about 25 mL³, about 0.5 mL³ to about 20 mL³, about 0.5 mL³ to about 15 mL³, about 0.5 mL³ to about 10 mL³, or about 0.5 mL³ to about 5 mL³. In some embodiments, the foulant has a volume of about 0.5 mL³, about 1 mL³, about 1.5 mL³, about 2 mL³, about 2.5 mL³, about 3 mL³, about 3.5 mL³, about 4 mL³, about 4.5 mL³, about 5 mL³, about 5.5 mL³, about 6 mL³, about 6.5 mL³, about 7 mL³, about 7.5 mL³, about 8 mL³, about 8.5 mL³, about 9 mL³, about 9.5 mL³, about 10 mL³, about 10.5 mL³, about 11 mL³, about 11.5 mL³, about 12 mL³, about 12.5 mL³, about 13 mL³, about 13.5 mL³, about 14 mL³, about 14.5 mL³, or about 15 mL³.

Once the composition contacts the foulant, the polymers of the foulant are dissolved and the dissolved polymer and components of the composition may be removed from the medium. In some embodiments, the components of the composition and dissolved polymer may flow with the medium, exit the component, and be collected, for example, in a waste container.

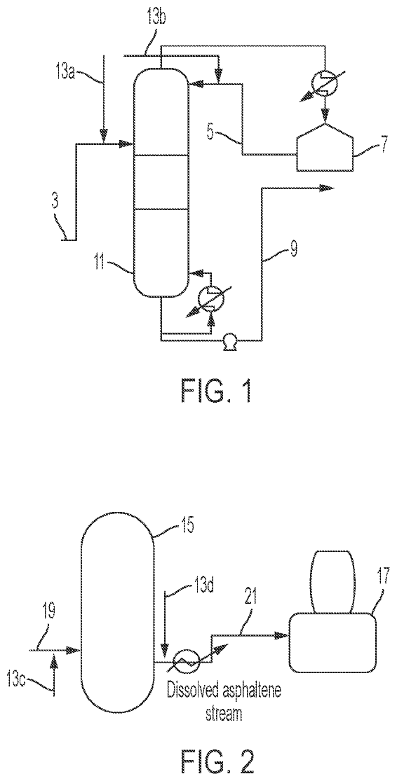

FIG. 1 depicts an example of the present disclosure including a water separation column 11. The column 11 may be part of a monomer manufacturing process, such as an acrylic acid manufacturing process. The composition may be injected 13a and/or 13b into the feed pipeline 3 or the toluene makeup pipeline 5 from a toluene tank 7, for example. The product from the water separation column leaves through the forerun column 9.

FIG. 2 depicts a spent acid tank 15, which stores spent acid and foulant, such as polymerized methacrylate from a methyl methacrylate manufacturing process. The foulant sinks to the bottom and blocks the pipeline 21 to the incinerator 17. However, the blockage can be dissolved/cleared by contacting it with a composition as disclosed herein. For example, the composition may be added at 13c and/or 13d into the feed stream 19 from a reactor, into the spent acid tank 15, and/or into the pipeline 21 to the incinerator 17.

Depending on the amount of foulant to be dissolved, about 10 ppm to about 10,000 ppm of the amide-containing compound is added to the medium. In some embodiments, the amide-containing compound may be added in an amount of about 10 ppm to about 1%, about 10 ppm to about 10,000 ppm, about 50 ppm to about 9,500 ppm, about 100 ppm to about 9,000 ppm, about 150 ppm, to 8,500 ppm, about 200 ppm to about 8,000 ppm, about 250 ppm to about 7,500 ppm, about 300 ppm to about 7,000 ppm, about 350 ppm to about 6,500 ppm, about 400 ppm to about 6,000 ppm, about 450 ppm to about 5,500 ppm, or about 500 ppm to about 5,000 ppm. In some embodiments, the amide-containing compound is added to the monomer manufacturing process in an amount of about 200 ppm to 2,000 ppm, or 500 ppm to 1,500 ppm. In an illustrative embodiment, the amide-containing compound may be added to a component and/or medium of the manufacturing process in an amount of about 100 ppm to 5,000 ppm. In some embodiments, the composition may be directly injected into the manufacturing process.

In some aspects, the amount of the amide-containing compound added to the monomer manufacturing process may be measured based on wt. % of the total medium present in either a single component (e.g., a recovery column or reboiler) or the entire monomer manufacturing process assembly. The amount of amide-containing compound added to the medium may be from about 0.5 wt. % to about 5 wt. %, about 0.75 wt. % to about 5 wt. %, about 1 wt. % to about 5 wt. %, about 1.25 wt. % to about 5 wt. %, about 1.5 wt. % to about 5 wt. %, about 1.75 wt. % to about 5 wt. %, about 2 wt. % to about 5 wt. %, about 2.25 wt. % to about 5 wt. %, about 2.5 wt. % to about 5 wt. %, about 2.75 wt. % to about 5 wt. %, or about 3 wt. % to about 5 wt. % of the medium.

In addition to the composition, the method may further include the addition of a polymerization inhibitor and/or a dispersant. In some embodiments, the composition comprises the polymerization inhibitor and/or the dispersant. Examples of polymerization inhibitors include, but are not limited to, phenolic compounds, amines, quinones, nitroxyl compounds and certain inorganic complexes (e.g., tetramethylpiperidinooxy, copper salts, phenothiazine (PTZ), hydroquinone (HQ) and monomethyl hydroquinone ether (MEHQ)). These inhibitors are designed to interrupt the polymerization reactions and prevent the formation of the polymer.

Dispersants are designed to adsorb on preexisting polymer particles, through chemical or physical interaction, and form an insulating layer on the polymer particles, which prevent the particles from agglomerating, thereby keeping them suspended in the process medium. Examples of dispersants include, but are not limited to, sulfonated hydrocarbons, such as dodecyl benzenesulfonate, poly isobutylene succinic acid esters, alkylphenolethoxylates, alkylphenolformaldehyde resins, fatty acid esters, fatty acid amides, fatty alcohol ethoxylates, polysaccharide esters, and the like.

The foregoing may be better understood by reference to the following examples, which are intended for illustrative purposes and are not intended to limit the scope of the disclosure or its application in any way.

### Examples

The ability of various compositions of the present disclosure to dissolve a foulant was tested. The compositions are described in Table 1 and Table 2.

TABLE 1

| Chemical Name | CAS | Weight Percentage |
|---|---|---|
| Dimethylacetamide | 127-19-5 | 30%-100% |
| Toluene | 108-88-3 | 0%-70% |

TABLE 2

| Chemical Name | CAS | Weight Percentage |
|---|---|---|
| Dimethylacetamide or | 127-19-5 | 30%-100% |
| Dimethylformamide | 68-12-2 | 30%-100% |
| Methanol | 67-56-1 | 0%-70% |

A foulant comprising acrylic acid polymer (referred to herein as acrylic acid foulant) was collected from a monomer manufacturing process. Two separate test tubes were set up and about 2 mL of the acrylic acid foulant, collected from an acrylic acid plant, was added to each tube. In the first test tube, about 10 mL of a composition containing dimethylacetamide was added. As a control, about 10 mL of a solution containing a tall oil fatty acid mixture was added to the second test tube. The first test tube showed that the dimethylacetamide was able to dissolve the foulant while the foulant in the second test tube remained aggregated and sank to the bottom of the tube. After allowing both test tubes to stand for 12 hours, the foulant remained dissolved in the first test tube.

Similar experiments were conducted using foulant comprising polymerized methyl methacrylate and other experiments were conducted using foulant comprising polymerized acrylonitrile.

These experiments showed that compositions comprising dimethyl phthalate and from about 30 wt. % to about 100 wt. % dimethylacetamide of the overall composition were highly effective at dissolving the foulant. In general, as the amount of dimethylacetamide added to the test tube increased, the amount of foulant dissolved increased. In some experiments, a composition containing a ratio of about 1:1 amide-containing compound and solvent efficiently dissolved the foulant.

Additionally, these experiments showed that compositions comprising dimethyl phthalate and from about 30 wt. % to about 100 wt. % dimethylformamide were highly effective at dissolving the foulant. In general, as the amount of dimethylformamide added to the test tube increased, the amount of foulant dissolved increased.

The experiments showed the effectiveness of the composition to dissolve foulant material produced from acrylic acid and remain dissolved when added back into a process medium of acrylic acid. In a first test tube, about 10 mL of acrylic acid was added. In a second test tube, about 10 ml of xylene was added. About 2.5 mL of acrylic acid foulant solution was added to each test tube. The acrylic acid foulant solution contained dimethylacetamide and about 2 mL of dissolved foulant. Even when added to a larger amount of the process medium (i.e., acrylic acid or xylene), the dissolved foulant did not precipitate out or form a sediment in either tube.

In some instances, the test tubes were shaken and in other instances, the test tubes were not shaken. While shaking appeared to increase the effectiveness of the composition, shaking was not necessary to dissolve the foulant.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a solvent” is intended to include “at least one solvent” or “one or more solvents.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25 °C with neat (not diluted) polymers.

As used herein, the term “about” refers to the cited value being within the errors arising from the standard deviation found in their respective testing measurements, and if those errors cannot be determined, then “about” may refer to, for example, within 5% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

1. A method of dissolving a foulant in a monomer manufacturing process, comprising:
adding a composition to a medium in the process, wherein the composition comprises an amide-containing compound and wherein the medium comprises the foulant,
contacting the amide-containing compound with the foulant, and
dissolving the foulant.

2. The method of claim 1, wherein the amide-containing compound comprises the following formula: 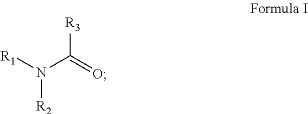
wherein R₁, R₂, and R₃ are independently selected from the group consisting of hydrogen, an alkyl group, an alkenyl group, and an aryl group.

3. The method of claim 2, wherein at least two of R₁, R₂, and R₃ are CH₃.

4. The method claim 1, wherein the amide-containing compound comprises the following formula:

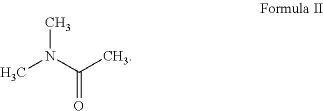

5. The method of claim 1, wherein the amide-containing compound has a weight average molecular weight from about 45 g/mol to about 300 g/mol.

6. The method of claim 1, wherein the composition comprises at least two different amide-containing compounds.

7. The method of claim 1, wherein the monomer is selected from the group consisting of acrylic acid, methacrylic acid, an acrylate, a methacrylic ester, acrylonitrile, and any combination thereof.

8. The method of claim 1, wherein the composition further comprises a solvent.

9. The method of claim 8, wherein the solvent is selected from the group consisting of a hydrocarbon, an ether, an ester, an alcohol, tetrahydrofuran, and any combination thereof.

10. The method of claim 8, wherein the solvent is selected from the group consisting of a hydrocarbon, tetrahydrofuran, an ether, and any combination thereof, and the monomer is acrylic acid.

11. The method of claim 8, wherein the solvent is selected from the group consisting of an alcohol, a hydrocarbon, and any combination thereof, and the monomer is selected from the group consisting of a methacrylic ester, an acrylate, and any combination thereof.

12. (canceled)

13. The method of claim 8, wherein the hydrocarbon is selected from toluene, kerosene, xylene, or any combination thereof.

14. The method of claim 8, wherein the composition comprises about 10 wt. % to about 90 wt. % of the amide-containing compound and about 90 wt. % to about 10 wt. % of the solvent.

15. The method of claim 1, wherein a component of the monomer manufacturing process comprises the medium.

16. The method of claim 15, wherein the component is selected from the group consisting of an absorption column, a stripper, a recovery column, a purification column, a product column, a reboiler, a spent acid tank, a conduit connecting any of the foregoing components, and any combination thereof.

17. (canceled)

18. The method of claim 1, wherein the composition is combined with an alkali wash water in a weight ratio of 1:10 to 1:1 and added to the medium.

19. The method of claim 1, wherein the composition and dissolved foulant are removed from the medium.

20. The method of claim 1, wherein about 10 ppm to about 10,000 ppm of the amide-containing compound is added to the medium.

21. The method of claim 1, wherein the medium comprises from about 0.5 wt. % to about 5 wt. % of the amide-containing compound.

22. The method of claim 1, wherein the composition further comprises a polymerization inhibitor and/or a dispersant.
